(19) United States
(12) Patent Application Publication
(10) Pub. No.: US 2023/0314401 A1
(43) Pub. Date: Oct. 5, 2023

(54) RESERVOIR FLUID IDENTIFICATION AND QUANTIFICATION USING TEMPERATURE TRANSIENT ANALYSIS

(71) Applicant: Saudi Arabian Oil Company, Dhahran (SA)

(72) Inventors: Ali J. Al Solial, Tarout Island (SA); Shouxiang Mark Ma, Dhahran (SA); Ali E. Alqunais, Al Jish (SA)

(21) Appl. No.: 17/657,038

(22) Filed: Mar. 29, 2022

(51) Int. Cl. G01N 33/28; E21B 49/08

(52) U.S. Cl. CPC G01N 33/2823; E21B 49/10; E21B 49/0875

(57) ABSTRACT

Example computer-implemented methods, media, and systems for reservoir fluid identification using temperature transient analysis are disclosed. One example method includes receiving formation pressure and temperature measurement data from formation testing of a first wellbore across a reservoir. A time period of steady-state formation temperature build-up during a formation pressure pre-test of the formation testing is determined. At least one of a cumulative rate of change, an instantaneous rate of change, or a combination of the two, of the formation temperature measurement data over the time period is determined. Formation fluid type of the first wellbore is determined based on at least one of the cumulative rate of change, the instantaneous rate of change, or a combination of the two, of the formation temperature measurement data. The determined formation fluid type is provided for prediction of formation fluid type of the first wellbore without formation fluid sampling of the first wellbore.

### TECHNICAL FIELD

The present disclosure relates to computer-implemented methods, media, and systems for reservoir fluid identification and quantification using temperature transient analysis.

### BACKGROUND

Reservoir saturating fluid type can be determined using formation sampling of a reservoir, which can involve high operational cost. On the other hand, formation pressure and temperature data are available during pretests of formation testing. The thermal quartz gauge used in modern formation testers has very high resolution, with a measurement error of around 0.009° F., and may be used to monitor small changes in formation temperature during pretests of formation testing.

### SUMMARY

The present disclosure involves computer-implemented methods, media, and systems for reservoir fluid identification and quantification using temperature transient analysis. One example computer-implemented method includes receiving formation pressure measurement data and formation temperature measurement data from formation testing of a first wellbore across a reservoir. A time period of steady-state formation temperature build-up during a single formation pressure pre-test of the formation testing is determined. At least one of a cumulative rate of change, an instantaneous rate of change, or a combination of the cumulative rate of change and the instantaneous rate of change, of the received formation temperature measurement data over the determined time period of steady-state formation temperature build-up is determined. Formation fluid type of the first wellbore is determined based on at least one of the cumulative rate of change, the instantaneous rate of change, or the combination of the cumulative rate of change and the instantaneous rate of change, of the received formation temperature measurement data, where the formation fluid type includes one of water and gas. The determined formation fluid type of the first wellbore is provided for prediction of formation fluid type of the first wellbore before or without formation fluid sampling of the first wellbore.

While generally described as computer-implemented software embodied on tangible media that processes and transforms the respective data, some or all of the aspects may be computer-implemented methods or further included in respective systems or other devices for performing this described functionality. The details of these and other aspects and implementations of the present disclosure are set forth in the accompanying drawings and the description below. Other features, objects, and advantages of the disclosure will be apparent from the description and drawings, and from the claims.

### DESCRIPTION OF DRAWINGS

FIG. 1 is a flowchart illustrating an example workflow for determining formation fluid type in a wellbore based on changes in formation temperature, in accordance with example implementations of this disclosure.

FIG. 2 illustrates an example formation tester that conducts a pre-test, in accordance with example implementations of this disclosure.

FIG. 3 illustrates an example of drawdown and build-up of formation pressure during the pre-test of formation testing, in accordance with example implementations of this disclosure.

FIG. 4 illustrates an example of build-up of formation temperature during a pre-test of formation testing, in accordance with example implementations of this disclosure.

FIG. 5 illustrates an example of rate of change in time and temperature against build-up time (in minutes) in a semi-log scale for applications of reservoir fluid typing, in accordance with example implementations of this disclosure.

FIG. 6 illustrates an example of the sensitivity of using cumulative temperature derivative for formation fluid typing, in accordance with example implementations of this disclosure.

FIG. 7 illustrates an example of the sensitivity of using Eqn. 3 for formation fluid typing, in accordance with example implementations of this disclosure.

FIG. 8 illustrates an example of establishing contrast between water and gas saturating zones of a reservoir, in accordance with example implementations of this disclosure.

FIG. 9 illustrates an example of determining reservoir fluid type using changes in formation temperature data, in accordance with example implementations of this disclosure.

FIG. 10 illustrates an example relationship between changes in thermal conductivity and sand sample's porosity or water filled porosity, in accordance with example implementations of this disclosure.

FIG. 11 illustrates an example of a lab study that shows the effect of saturating fluid on rate of temperature change, in accordance with example implementations of this disclosure.

FIG. 12 is a flowchart illustrating an example of a method for reservoir fluid identification using temperature transient analysis, in accordance with example implementations of this disclosure.

FIG. 13 is a schematic illustration of example computer systems that can be used to execute implementations of the present disclosure.

### DETAILED DESCRIPTION

Phenomena of effects of rock properties (mineralogy, porosity, and saturation) on thermal heat transfer have many applications, one example application is fluid typing during formation testing and sampling in environments such as tight formations or fresh water environment.

With the advancements in thermal gauges, there lies a potential to utilize formation temperature measurements from high resolution thermal gauges, for example, thermal quartz gauges, to derive valuable information about formation fluid type, without formation sampling.

This disclosure describes technologies for determining formation fluid type with measurements in a wellbore, based on changes in high resolution formation temperature measurement from pretests of formation testing. In some implementations, the changes in formation temperature are caused by pumping-out and fluid expansion during operational in situ formation testing. When formation evaluation results from mud logging (such as mud gas) and resistivity measurement are not reliable, such as in tight reservoirs where resistivity log quality may be affected by low permeability and porosity, resistivity log based saturation calculation carries large uncertainties. Moreover, determining formation fluid type using formation temperature measurement can assist in evaluating formations where water salinity is low, i.e., the industrywide challenge of formation evaluation in fresh water environment, where the small conductivity contrast between fresh water and hydrocarbons limits applications of resistivity-based formation evaluation.

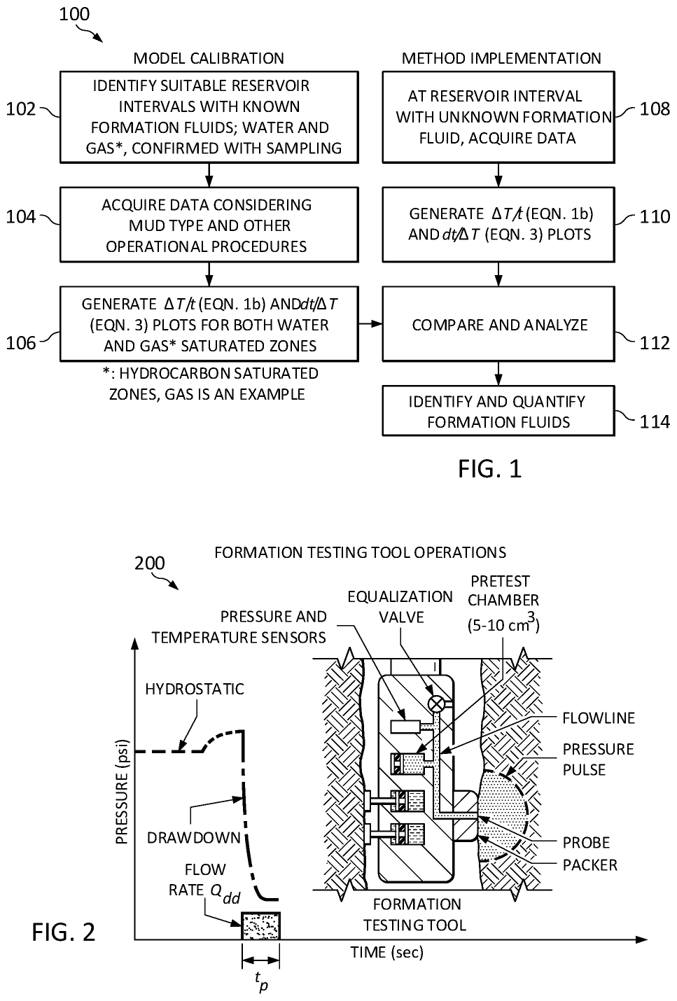

FIG. 1 is a flowchart illustrating an example workflow 100 for determining formation fluid type in a wellbore based on changes in formation temperature, in accordance with example implementations of this disclosure.

At step 102, reservoir intervals with known formation fluids of water and gas are identified. These identified reservoir intervals provide, respectively, data from water and gas reservoir intervals that are used to determining benchmark changes in temperature over time, which will be used for comparison with changes in temperature over time across reservoir intervals where formation fluid type is to be determined based on the comparison. The changes in temperature over time can include, for example, ΔT/t and dt/ΔT as defined in Eqns. 1b and 3 respectively. Water and gas in the identified reservoir intervals are confirmed by water and gas samples sampled from the respective reservoir intervals.

At step 104, formation pressure and temperature data are extracted from the same wellbore or two wellbores, e.g., a first wellbore and a second wellbore, during pressure tests. Mud type and other operational procedures can be considered at this step.

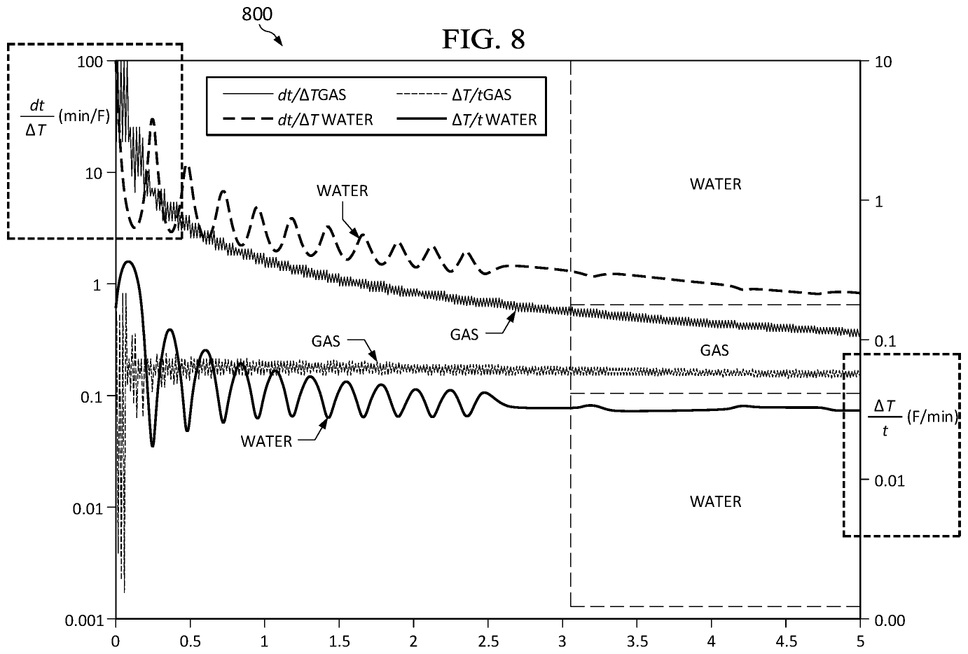

At step 106, ΔT/t (Eqn. 1b) and dt/ΔT (Eqn. 3) are generated on the same graph using formation pressure and temperature data extracted at step 104. An example of the resulting graph is shown in FIG. 8.

At step 108, formation pressure and temperature data are extracted from either the same wellbore or a different wellbore of a reservoir interval with unknown formation fluid type that is to be determined based on the extracted formation pressure and temperature data.

At step 110, ΔT/t and dt/ΔT are generated using formation pressure and temperature data extracted at step 108.

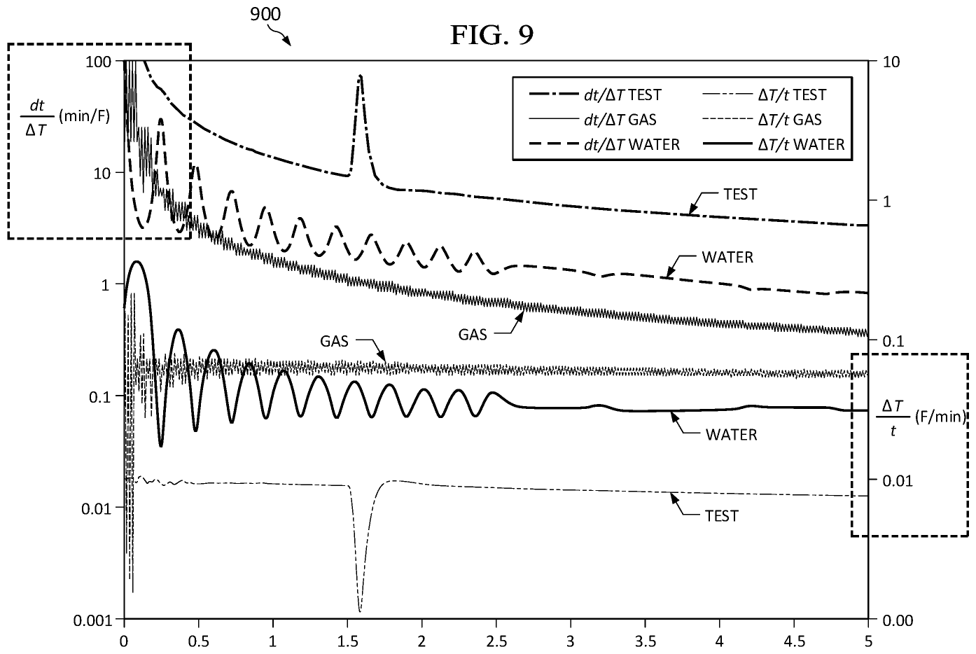

At step 112, ΔT/t and dt/ΔT generated at step 106 are compared with ΔT/t and dt/ΔT generated at step 110, and the comparison result is analyzed for the formation fluid type of the unknown formation fluid type reservoir interval. An example comparison result is shown in FIG. 9.

At step 114, the formation fluid type of the unknown formation fluid type reservoir interval is identified. As shown in FIG. 9, the example formation fluid type of unknown formation fluid type reservoir interval (corresponding to the two curves labeled “test”) is identified to be water. This example result can be further verified by physically sampling the formation used in the example of FIG. 9.

FIG. 2 illustrates an example formation tester 200 that conducts a pre-test. Formation testing and sampling with a formation tester is a formation evaluation operation that can incur high cost. It has two basic operation modes. One mode is formation testing, which is an operation measured in minutes for each test. For example a pre-test can take less than 10 minutes for a 10 and (milli-Darcy)/cp (centipoise) formation. Another mode is formation sampling, which is a process that can take several hours in order to sample clean formation fluid free of drilling fluid filtrate contamination. During formation testing, using pressure and thermal gauges, reservoir intervals are surveyed along a wellbore with measurements of formation pressure and temperature. The aforementioned formation testing with measurements of formation pressure is called pre-test. While formation pressure is an measurement that can be used in several areas, for example, (i) for evaluation of reservoir potential, because the higher the reservoir pressure, the higher the reserve potential, especially for gas reservoirs because gas is highly compressible, (ii) estimation of formation mobility and permeability, (iii) formation fluid typing with multiple pre-tests across a true vertical reservoir interval, because formation pressure gradients, such as in psi per true vertical feet, are proportional to fluid density, for example, 0.433 psi/ft for fresh water, 0.3 psi/ft for oil, and 0.1 psi/ft for gas (depending on pressure because gas is highly compressible), and (iv) free water level determination, formation temperature from a single pre-test can be used to determine the type of the formation fluid, in accordance with example implementations of this disclosure.

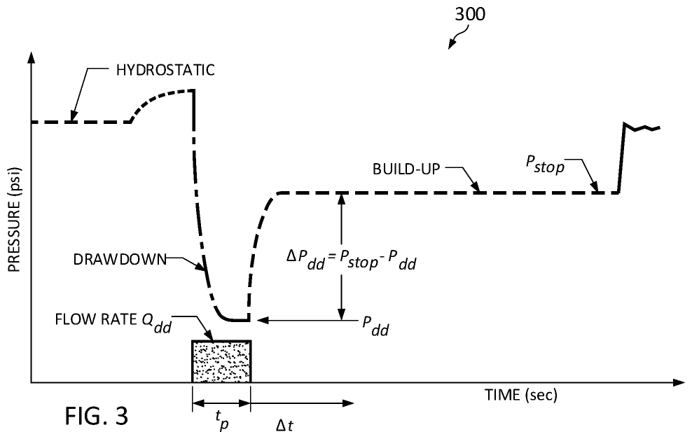

FIG. 3 illustrates an example 300 of drawdown and build-up of formation pressure during the pre-test of formation testing, in accordance with example implementations of this disclosure. In some implementations, during formation testing and sampling, the formation is subject to a drawdown, that is, the formation is pumped (i.e., turning on the pump to withdraw fluid from formation, see for example FIG. 2). This drawdown operation results in a formation pressure drop, which is accompanied by a formation temperature drop initially but then build-up steadily due to the hotter formation fluid flowing from the formation to the formation tester during pumping. After the drawdown, a build-up of formation pressure and a faster build-up of formation temperature follow, after the pump is turned off (see for example FIG. 3). As such the rock/fluid system enters a heating phase where thermal energy flows from the formation towards the formation tester's gauges, contacted to the wellbore sand-face.

In some implementations, thermal quartz gauges are used in formation testers and have high resolution, i.e., thermal quartz gauges are sensitive to small changes in temperature, with a measurement error of around 0.009° F. Therefore they can be used to monitor small changes in temperature during formation testing pretests. The small change in temperature as a function of formation fluid types enables fluid typing based on changes in formation temperature.

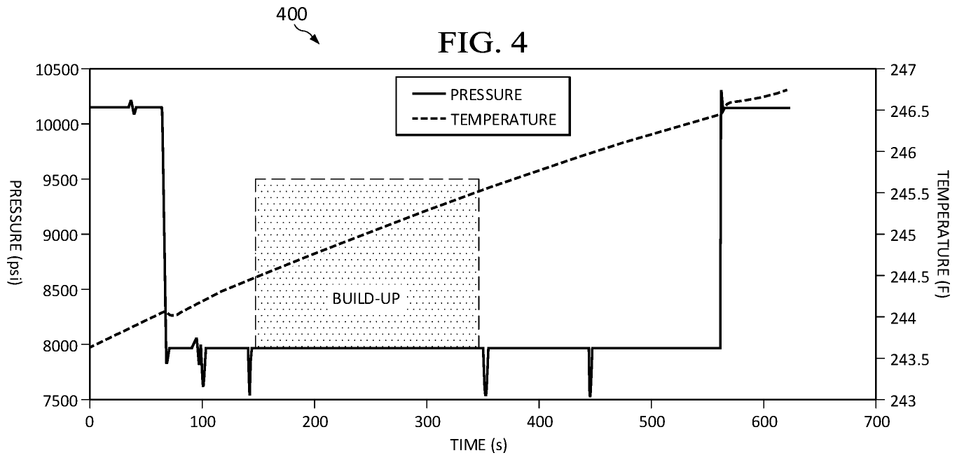

FIG. 4 illustrates an example 400 of build-up of formation pressure and temperature during a pre-test of formation testing, in accordance with example implementations of this disclosure. In some implementations, a stabilized pressure drawdown time period during formation testing is determined, when temperature started to build-up. This can be done by first plotting formation pressure (the pressure curve in FIG. 4) and temperature as functions of time, and then identifying the start and end of the build-up period of temperature. FIG. 4 shows the period of the targeted temperature build-up steadily (shaded period). Note that in FIG. 4, the formation temperature (the temperature curve in FIG. 4) changes over time, by more than 1° F., during a time period when there is also pressure change, i.e., pressure drawdown is stabilized. This time period is then used to create plots of changes in time and formation temperature, as described next.

In some implementations, by taking the reading from a thermal gauge against time, a temperature build-up behavior can be constructed using the instant temperature derivative:

$$\frac{dT}{dt} = \frac{\vert {T}_{n} - {T}_{n - 1} \vert}{{t}_{n} - {t}_{n - 1}} , \qquad ( 1 a )$$

or inverse of it

$$\frac{dt}{dT} = \frac{{t}_{n} - {t}_{n - 1}}{\vert {T}_{n} - {T}_{n - 1} \vert} , \qquad ( 2 a )$$

where T denotes formation temperature measurement at a data point, t denotes the time at a data point, and the subscripts n−1 and n denotes the (n−1)th and nth data points respectively.

In some implementations, the temperature build-up behavior can also be constructed by considering the overall trend in the rate of temperature change within the identified temperature transient period:

$$\frac{Δ T}{t} = \frac{\vert {T}_{n} - {T}_{i} \vert}{{t}_{n} - {t}_{i}} , \qquad ( 1 b )$$

or inverse of it

$$\frac{t}{Δ T} = \frac{{t}_{n} - {t}_{i}}{\vert {T}_{n} - {T}_{i} \vert} , \qquad ( 2 b )$$

where ΔT denotes the temperature change between the nth data point and the initial data point of the selected build-up period, t denotes the time change between the nth data point and the initial data point of the selected build-up period, and the subscript i denotes the initial data point of the selected build-up period.

In some implementations, after rearranging the parameters in Eqns. 1a to 2b, a form of mixed expression of instant and cumulative changes in time and temperature can also be expressed as a function of fluid typing, as shown in the equation below:

$$\frac{d t}{Δ T} = \frac{{t}_{n} - {t}_{n - 1}}{\vert {T}_{n} - {T}_{i} \vert} . \qquad ( 3 )$$

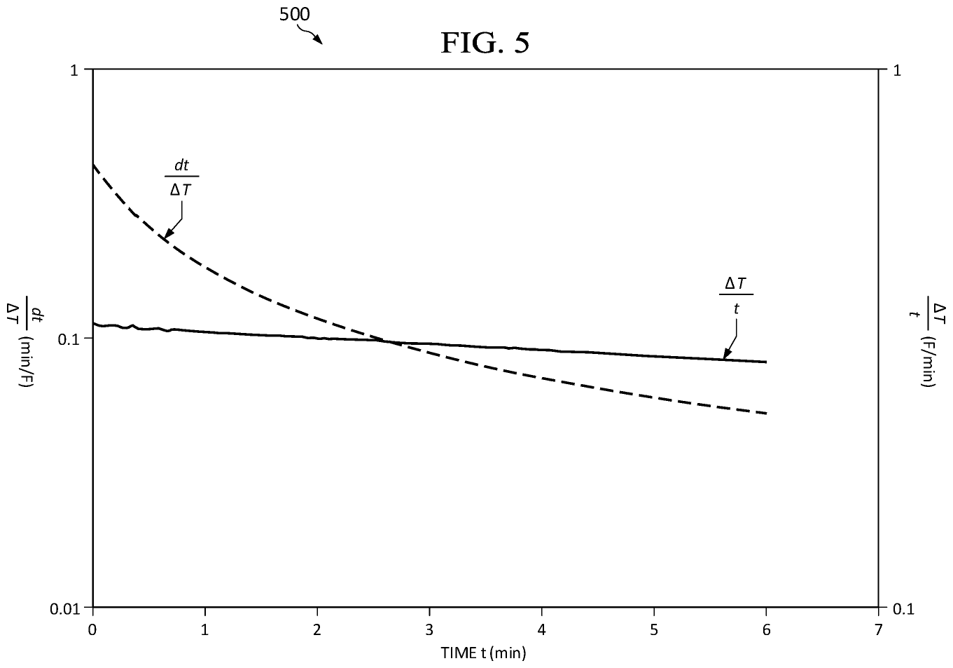

FIG. 5 illustrates an example 500 of changes in time per unit temperature and temperature per unit time against build-up time (in minutes) in a semi-log scale for applications of reservoir fluid typing, in accordance with example implementations of this disclosure. In some implementations, thermal transient in porous media depends on the type and amount of saturating fluids. Data points with the same saturation, for example, the same water saturation, will stack, while points with different saturations, for example, different water saturations, will show different responses, in terms of rate of temperature change per unit time and time change per unit temperature. The resulting graph will have reservoir fluid types classified and reservoir saturation qualified. Therefore both temperature and temperature derivatives (Eqns. 1a to 3) can be used in temperature transient analysis for reservoir fluid typing.

In some implementations, by comparing the instant rate of temperature change with time at a specific time tₙ (Eqns. 1a and 2a) with the cumulative rate of temperature change over the time period from the initial data point of the build-up to a specific time tₙ, (Eqns. 1b and 2b), it is noted that:
The instant rate of temperature change with time (Eqns. 1a and 2a) is more sensitive to changes in temperature. This can be used to identify finer features of thermal behavior shown by the rock/fluid system.
The cumulative rate of change in temperature over time (Eqns. 1b, 2b, and 3) is more stable in predicting macroscopic changes in the rock/fluid system, such as fluid typing and quantification.

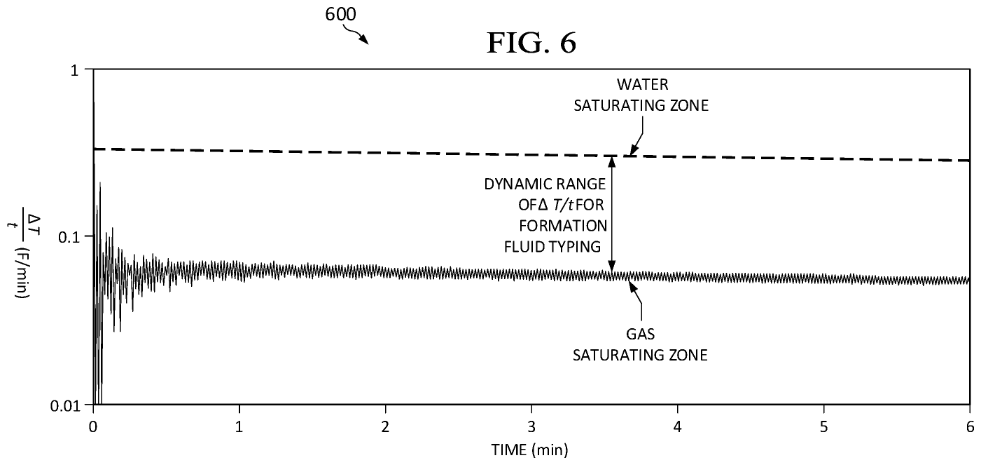

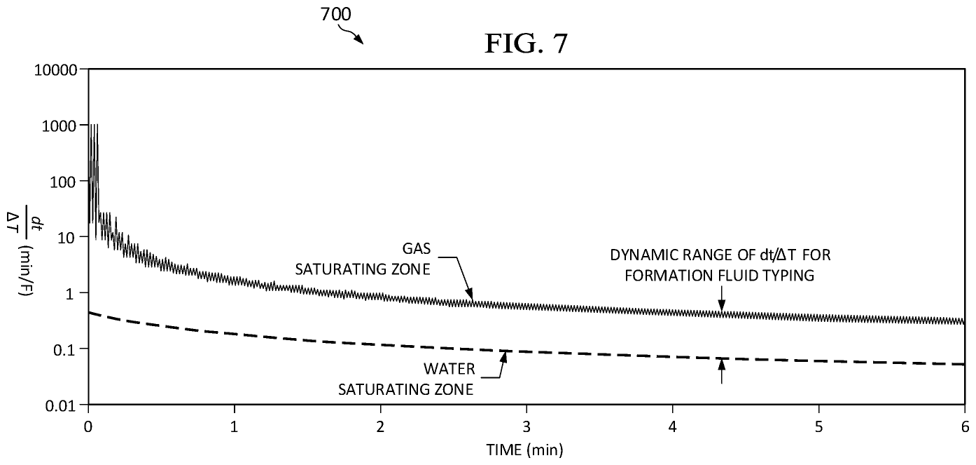

FIG. 6 illustrates an example 600 of the sensitivity of using cumulative temperature derivative (Eqn. 1b) for formation fluid typing, in accordance with example implementations of this disclosure. FIG. 7 illustrates an example 700 of the sensitivity of using Eqn. 3 for formation fluid typing, in accordance with example implementations of this disclosure. In some implementations, the plots in FIG. 5 can be used to compare the responses from different saturation conditions. With data points from formation fluid samples taken in a field, Eqns. 1b and 3 can have dynamic range that can be used to differentiate between water and gas saturating zones, as shown in FIG. 6 and FIG. 7.

FIG. 6 and FIG. 7 show the contrast between reservoir intervals saturated with water and gas. In some implementations, this contrast can be established before the study of thermal stability. Measures should be taken to ensure that the drilling fluid's interference on temperature diffusivity does not mask the effect of formation fluid saturation. Moreover, the process of formation fluid typing based on changes in formation temperature, as described in this disclosure, provides a way to check the quality of data acquired in terms of potential issues with the gauge or the tool.

FIG. 7 shows the contrast between water and gas saturating reservoir intervals for dt/ΔT (Eqn. 3). In some implementations, similar to the graph in FIG. 6 that shows the contrast between water and gas saturating intervals for ΔT/t (Eqn. 1b), the graph in FIG. 7 can also be used to differentiate between water and gas saturating zones, and it can be combined with other tools to analyze the rock behavior and points of similar saturating fluid to look for differences in thermal behavior.

In some implementations, instead of using thermal stability based tools, graphs generated based on Eqns. 1b and 3 can be used as tools for fluid typing. However, the thermal stability and the average rate of change in temperature can be used as a quick guideline to determine the saturating fluid. This guideline is the radial diffusivity equation:

$$\frac{\partial T}{\partial t} = k [ \frac{{\partial }^{2} T}{\partial {r}^{2}} + \frac{1}{r} \frac{\partial T}{\partial r} ] . \qquad ( 4 )$$

Thermal stability and average rate of change in temperature with respect to time correspond to ∂T/∂t from the radial diffusivity equation and as such they are directly affected by the thermal diffusivity of the rock/fluid system, and show the saturation change when diffusivity of mud is higher than at least one of the fluids saturating the formation.

In some implementations, to ensure that Eqns. 1b and 3 have dynamic range that can be used to differentiate between water and gas saturating zones for the reservoir to be tested, contrast threshold between water and gas saturating zones needs to be established first, in order to avoid cases where the contrast between water and gas saturating zones is too low to differentiate between water and gas saturating zones for the reservoir to be tested. Low contrast cases can occur when the diffusivities of water and hydrocarbon are close to each other.

In some implementations, the pressure and temperature data points in the formation testing are taken from the point closest to the surface (the shallowest depth) to the point furthest from the surface (the deepest) to avoid the mud cooling the gauge instead of the formation providing the thermal support. Therefore formation pre-testing is moving deeper along the wellbore depth progressively.

In some implementations, behaviors with both water based mud (WBM) and oil based mud (OBM) need to be investigated, so that mud selection can be a way of finding oil and gas with analysis of heat transient.

Formation testing and sampling are logging operations that can incur high cost. Identifying formation fluid types from temperature transient analysis, as described in the disclosure, before time consuming sampling or without time consuming sampling, can lead to reduction in operational cost, better well completion, and better well/reservoir performance.

An example relationship between reservoir fluid type and changes in formation temperature is described next.

In some implementations, at constant pressure, thermal diffusivity, K, is a function of thermal conductivity (λ), normalized with density (ρ) and specific heat capacity (h_{c}). Thermal diffusivity is a measurement of an object's ability to transfer heat, i.e., the rate of heat transfer of a material. The higher the object's K, the more resistant there will be to changes in equilibrium as the object will be able to redistribute changes in temperature faster. In other words, in a substance with higher thermal diffusivity, heat moves more rapidly through the substance because the substance conducts heat more quickly relative to its volumetric heat capacity. This is shown in the below equation and dimensional analysis:

$$K = \frac{λ [ \frac{W}{m \cdot K} ]}{ρ [ \frac{kg}{{m}^{3}} ] {h}_{c} [ \frac{J}{kg \cdot K} ]} = \frac{λ [ \frac{W}{m \cdot K} ]}{{v}_{h c} [ \frac{J}{{m}^{3} K} ]} = \frac{λ [ \frac{W}{m \cdot K} ]}{{v}_{h c} [ \frac{W \cdot s}{{m}^{3} K} ]} = [ \frac{{m}^{2}}{s} ] \qquad ( 5 )$$

Note that the product of material density and specific heat capacity is expressed as the volumetric heat capacity (v_{hc}).

Thermal diffusivity measures the rate of heat propagation as area/time (m²/s). In other words, the continuous temperature response is controlled by thermal diffusivity. When an object's temperature decreases or increases, the object redistributes the energy within it to return to equilibrium. If a system has infinite thermal capacity (i.e., v_{hc}→∞), the thermal diffusivity becomes zero (i.e., K→0), that is, the system always returns to its original temperature. However, even in the case of an infinite thermal capacity, the system needs an equally large conductivity (λ) for that thermal capacity to move around. Therefore thermal equilibrium is controlled by diffusivity.

A system composed of multiple components has its conductivity as a combination of the conductivities of its components, following a mixing rule augmented by porosity ϕ for a porous media system. The following equation shows the conductivity in porous systems:

λₑ=λₛ^{(1-Ø)}λ_{f}^{Ø}, (6)

where subscript e denotes the effective measurement of the total system, s denotes the solid matrix, and f denotes the saturating fluid in the formation of the reservoir. If the solid matrix remains the same, the change in conductivity is a result of the change in the saturating fluid.

Heat capacity has a similar relationship but percentages differ based on whether it is volumetric or mass heat capacity.

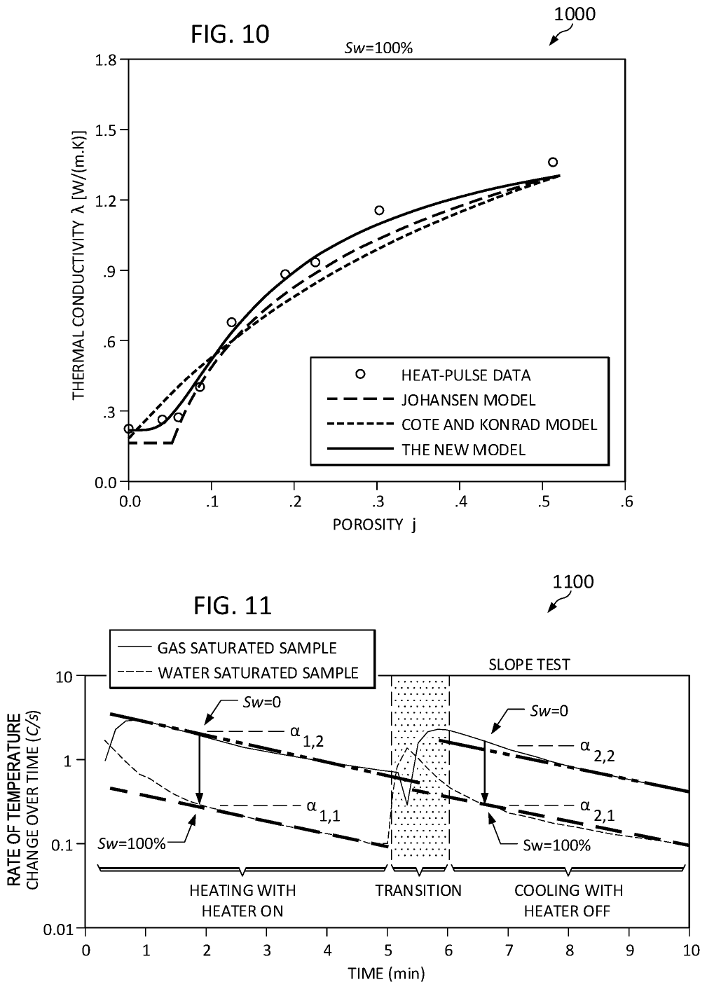

FIG. 10 illustrates an example relationship 1000 between changes in thermal conductivity and sand sample's porosity saturated 100% with water, in accordance with example implementations of this disclosure. It shows that thermal conductivity increases with sand samples' porosity or water filled porosity.

FIG. 11 illustrates an example 1100 of a laboratory study that shows the effect of saturating fluid on rate of temperature change, in accordance with example implementations of this disclosure. The laboratory study uses a dual needle probe, KDA 2 pro using SH-3 probe, inside two porous media samples, with one saturated with water, and the other saturated with air and dried in an oven at 70° C. From FIG. 11, the rate of temperature change with time, temperature derivative, (dT/dt in ° C./s) changes when the fluid inside each sample changes. The slope of dT/dt vs time t, also denoted as a, representing acceleration (during heating) or de-acceleration (during cooling), in ° C./s², indicates how fast the rate of dT/dt would change at each condition of dry (S_{w}=0, Sₐᵢᵣ=100%) and wet (S_{w}=100%), where S_{w} denotes water saturation, and Sₐᵢᵣ denotes air saturation. FIG. 11 shows that pore fluid type affects dT/dt.

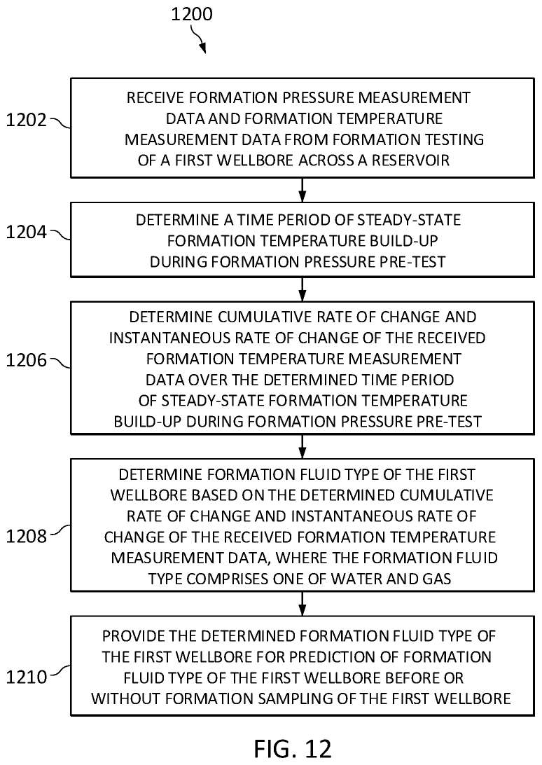

FIG. 12 illustrates an example 1200 of a method for reservoir fluid identification using temperature transient analysis, in accordance with example implementations of this disclosure.

At 1202, a computer system receives formation pressure measurement data and formation temperature measurement data from formation testing of a first wellbore across a reservoir.

At 1204, the computer system determines a time period of steady-state formation temperature build-up during formation pressure pre-test.

At 1206, the computer system determines a cumulative rate of change, an instantaneous rate of change, or a combination of the cumulative rate of change and the instantaneous rate of change such as Eqn. 3, of the received formation temperature measurement data over the determined time period of steady-state formation temperature build-up during formation pressure pre-test.

At 1208, the computer system determines formation fluid type of the first wellbore based on the determined cumulative rate of change, the instantaneous rate of change, or the combination of the cumulative rate of change and the instantaneous rate of change such as Eqn. 3, of the received formation temperature measurement data, where the formation fluid type includes one of water and gas.

At 1210, the computer system provides the determined formation fluid type of the first wellbore for prediction of formation fluid type of the first wellbore before or without formation fluid sampling of the first wellbore.

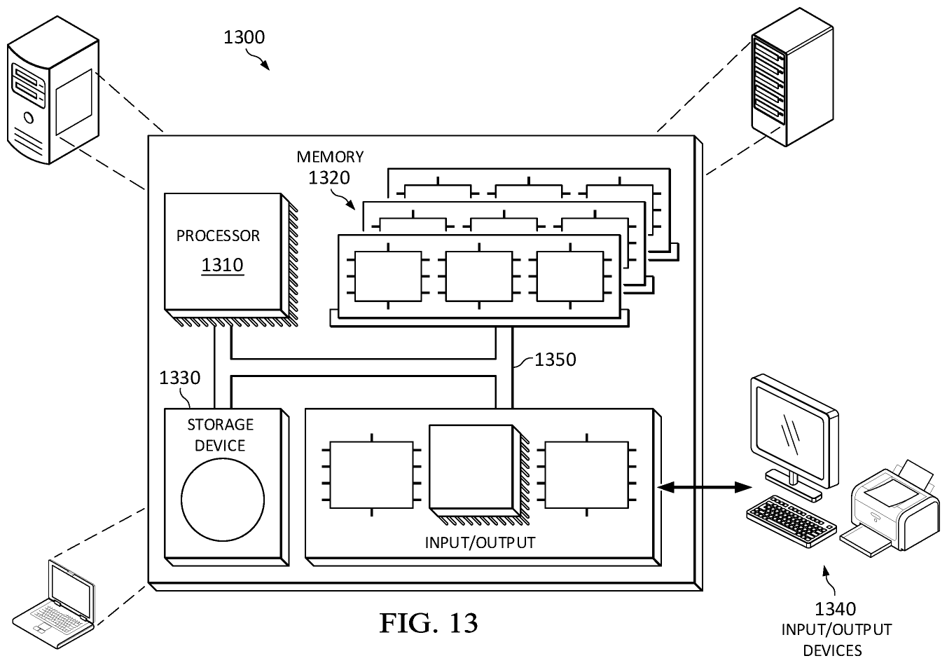

FIG. 13 illustrates a schematic diagram of an example computing system 1300. The system 1300 can be used for the operations described in association with the implementations described herein. For example, the system 1300 may be included in any or all of the server components discussed herein. The system 1300 includes a processor 1310, a memory 1320, a storage device 1330, and an input/output device 1340. The components 1310, 1320, 1330, and 1340 are interconnected using a system bus 1350. The processor 1310 is capable of processing instructions for execution within the system 1300. In some implementations, the processor 1310 is a single-threaded processor. The processor 1310 is a multi-threaded processor. The processor 1310 is capable of processing instructions stored in the memory 1320 or on the storage device 1330 to display graphical information for a user interface on the input/output device 1340.

The memory 1320 stores information within the system 1300. In some implementations, the memory 1320 is a computer-readable medium. The memory 1320 is a volatile memory unit. The memory 1320 is a non-volatile memory unit. The storage device 1330 is capable of providing mass storage for the system 1300. The storage device 1330 is a computer-readable medium. The storage device 1330 may be a floppy disk device, a hard disk device, an optical disk device, or a tape device. The input/output device 1340 provides input/output operations for the system 1300. The input/output device 1340 includes a keyboard and/or pointing device. The input/output device 1340 includes a display unit for displaying graphical user interfaces.

Certain aspects of the subject matter described here can be implemented as a method. Formation pressure measurement data and formation temperature measurement data are received from formation testing of a first wellbore across a reservoir. A time period of steady-state formation temperature build-up during formation pressure pre-test is determined. At least one of a cumulative rate of change, an instantaneous rate of change, or a combination of the cumulative rate of change and the instantaneous rate of change such as Eqn. 3, of the received formation temperature measurement data is determined over the determined time period of steady-state formation temperature build-up during a single formation pressure pre-test. Formation fluid type of the first wellbore is determined based on at least one of the cumulative rate of change, the instantaneous rate of change, or the combination of the cumulative rate of change and the instantaneous rate of change such as Eqn. 3, of the received formation temperature measurement data. The formation fluid type includes one of water and gas. The determined formation fluid type of the first wellbore is provided for prediction of formation fluid type of the first wellbore before or without formation fluid sampling of the first wellbore.

An aspect taken alone or combinable with any other aspect includes the following features. The determining at least one of the cumulative rate of change, the instantaneous rate of change, or the combination of the cumulative rate of change and the instantaneous rate of change, of the received formation temperature measurement data over the determined time period of steady-state formation temperature build-up includes determining

$$\frac{{t}_{n} - {t}_{n - 1}}{\vert {T}_{n} - {T}_{i} \vert} \qquad ( Eq . 3 )$$

for all data points of the received formation temperature measurement data over the determined time period of steady-state formation temperature steady-state formation temperature build-up, where tₙ₋₁ and tₙ represent respectively time instants of (n−1)th data point and nth data point of the received formation temperature measurement data over the determined time period of steady-state formation temperature build-up, Tₙ represents nth data point value of the received formation temperature measurement data over the determined time period of steady-state formation temperature build-up, and Tᵢ represents the initial data point value of the received formation temperature measurement data over the determined time period of steady-state formation temperature build-up.

An aspect taken alone or combinable with any other aspect includes the following features. The determining at least one of the cumulative rate of change, the instantaneous rate of change, or the combination of the cumulative rate of change and the instantaneous rate of change, of the received formation temperature measurement data over the determined time period of steady-state formation temperature build-up includes determining

$$\frac{\vert {T}_{n} - {T}_{i} \vert}{{t}_{n} - {t}_{i}} \qquad ( Eq . 1 b )$$

for all data points of the received formation temperature measurement data over the determined time period of steady-state formation temperature build-up, where tₙ and tᵢ represent respectively time instants associated with nth data point and initial data point of the received formation temperature measurement data over the determined time period of steady-state formation temperature build-up, Tₙ represents the nth data point of the received formation temperature measurement data over the determined time period of steady-state formation temperature build-up, and Tᵢ represents the initial data point of the received formation temperature measurement data over the determined time period of steady-state formation temperature build-up.

An aspect taken alone or combinable with any other aspect includes the following features. Before determining the time period of steady-state formation temperature build-up, establishing a baseline cumulative rate of change of water temperature, a baseline instantaneous rate of change of water temperature, a baseline combination of cumulative and instantaneous rates of change of water temperature, a baseline cumulative rate of change of gas temperature, a baseline instantaneous rate of change of gas temperature, and a baseline combination of cumulative and instantaneous rates of change of gas temperature, by using temperature measurement data from a wellbore across multiple reservoir intervals, where at least one of the multiple reservoir intervals has a known formation fluid type of water, and at least one of the multiple reservoir intervals has a known formation fluid type of gas.

An aspect taken alone or combinable with any other aspect includes the following features. The determining the formation fluid type based on at least one of the cumulative rate of change, the instantaneous rate of change, or the combination of the cumulative rate of change and the instantaneous rate of change, of the received formation temperature measurement data over the determined time period of steady-state formation temperature build-up includes at least one of comparing the cumulative rate of change of the received formation temperature measurement data with the baseline cumulative rate of change of water temperature and the baseline cumulative rate of change of gas temperature, comparing the determined instantaneous rate of change of the received formation temperature measurement data with the baseline instantaneous rate of change of water temperature and the baseline instantaneous rate of change of gas temperature, or comparing the determined combination of the cumulative rate of change and the instantaneous rate of change of the received formation temperature measurement data with the baseline combination of cumulative and instantaneous rates of change of water temperature and the baseline combination of cumulative and instantaneous rates of change of gas temperature.

An aspect taken alone or combinable with any other aspect includes the following features. The formation temperature measurement data includes data from a first temperature measurement point in formation testing at a first depth and data from a second temperature measurement point in formation testing at a second depth, where the second depth is deeper or larger than the first depth.

An aspect taken alone or combinable with any other aspect includes the following features. The determining at least one of the cumulative rate of change, the instantaneous rate of change, or the combination of the cumulative rate of change and the instantaneous rate of change, of the received formation temperature measurement data over the determined time period of steady-state formation temperature build-up includes determining

$$\frac{\vert {T}_{n} - {T}_{n - 1} \vert}{{t}_{n} - {t}_{n - 1}} or \qquad ( Eqn . 1 a )$$

$$\frac{{t}_{n} - {t}_{n - 1}}{\vert {T}_{n} - {T}_{n - 1} \vert} \qquad ( Eqn . 2 a )$$

for all data points of the received formation temperature measurement data over the determined time period of steady-state formation temperature build-up, where tₙ and tₙ₋₁ represent respectively time instants associated with nth data point and (n−1)th data point of the received formation temperature measurement data over the determined time period of steady-state formation temperature build-up, and where Tₙ and Tₙ₋₁ represent respectively the nth data point and the (n−1)th data point of the received formation temperature measurement data over the determined time period of steady-state formation temperature build-up.

Certain aspects of the subject matter described in this disclosure can be implemented as a non-transitory computer-readable medium storing instructions which, when executed by a hardware-based processor perform operations including the methods described here.

Certain aspects of the subject matter described in this disclosure can be implemented as a computer-implemented system that includes one or more processors including a hardware-based processor, and a memory storage including a non-transitory computer-readable medium storing instructions which, when executed by the one or more processors performs operations including the methods described here.

The features described can be implemented in digital electronic circuitry, or in computer hardware, firmware, software, or in combinations of them. The apparatus can be implemented in a computer program product tangibly embodied in an information carrier (e.g., in a machine-readable storage device, for execution by a programmable processor), and method operations can be performed by a programmable processor executing a program of instructions to perform functions of the described implementations by operating on input data and generating output. The described features can be implemented advantageously in one or more computer programs that are executable on a programmable system including at least one programmable processor coupled to receive data and instructions from, and to transmit data and instructions to, a data storage system, at least one input device, and at least one output device. A computer program is a set of instructions that can be used, directly or indirectly, in a computer to perform a certain activity or bring about a certain result. A computer program can be written in any form of programming language, including compiled or interpreted languages, and it can be deployed in any form, including as a stand-alone program or as a module, component, subroutine, or other unit suitable for use in a computing environment.

Suitable processors for the execution of a program of instructions include, by way of example, both general and special purpose microprocessors, and the sole processor or one of multiple processors of any kind of computer. Generally, a processor will receive instructions and data from a read-only memory or a random access memory or both. Elements of a computer can include a processor for executing instructions and one or more memories for storing instructions and data. Generally, a computer can also include, or be operatively coupled to communicate with, one or more mass storage devices for storing data files; such devices include magnetic disks, such as internal hard disks and removable disks; magneto-optical disks; and optical disks. Storage devices suitable for tangibly embodying computer program instructions and data include all forms of non-volatile memory, including by way of example semiconductor memory devices, such as EPROM, EEPROM, and flash memory devices; magnetic disks such as internal hard disks and removable disks; magneto-optical disks; and CD-ROM and DVD-ROM disks. The processor and the memory can be supplemented by, or incorporated in, ASICs (application-specific integrated circuits).

To provide for interaction with a user, the features can be implemented on a computer having a display device such as a cathode ray tube (CRT) or liquid crystal display (LCD) monitor for displaying information to the user and a keyboard and a pointing device such as a mouse or a trackball by which the user can provide input to the computer.

The features can be implemented in a computer system that includes a back-end component, such as a data server, or that includes a middleware component, such as an application server or an Internet server, or that includes a front-end component, such as a client computer having a graphical user interface or an Internet browser, or any combination of them. The components of the system can be connected by any form or medium of digital data communication such as a communication network. Examples of communication networks include, for example, a LAN, a WAN, and the computers and networks forming the Internet.

The computer system can include clients and servers. A client and server are generally remote from each other and typically interact through a network, such as the described one. The relationship of client and server arises by virtue of computer programs running on the respective computers and having a client-server relationship to each other.

In addition, the logic flows depicted in the figures do not require the particular order shown, or sequential order, to achieve desirable results. In addition, other operations may be provided, or operations may be eliminated, from the described flows, and other components may be added to, or removed from, the described systems. Accordingly, other implementations are within the scope of the following claims.

The preceding figures and accompanying description illustrate example processes and computer-implementable techniques. But system 100 (or its software or other components) contemplates using, implementing, or executing any suitable technique for performing these and other tasks. It will be understood that these processes are for illustration purposes only and that the described or similar techniques may be performed at any appropriate time, including concurrently, individually, or in combination. In addition, many of the operations in these processes may take place simultaneously, concurrently, and/or in different orders than as shown. Moreover, system 100 may use processes with additional operations, fewer operations, and/or different operations, so long as the methods remain appropriate.

In other words, although this disclosure has been described in terms of certain implementations and generally associated methods, alterations and permutations of these implementations and methods will be apparent to those skilled in the art. Accordingly, the above description of example implementations does not define or constrain this disclosure. Other changes, substitutions, and alterations are also possible without departing from the spirit and scope of this disclosure.

What is claimed is:

1. A computer-implemented method, comprising:
receiving formation pressure measurement data and formation temperature measurement data from formation testing of a first wellbore across a reservoir;
determining a time period of steady-state formation temperature build-up during a single formation pressure pre-test of the formation testing;
determining at least one of a cumulative rate of change, an instantaneous rate of change, or a combination of the cumulative rate of change and the instantaneous rate of change, of the received formation temperature measurement data over the determined time period of steady-state formation temperature build-up;
determining formation fluid type of the first wellbore based on at least one of the cumulative rate of change, the instantaneous rate of change, or the combination of the cumulative rate of change and the instantaneous rate of change, of the received formation temperature measurement data over the determined time period of steady-state formation temperature build-up, wherein the formation fluid type comprises one of water and gas; and
providing the determined formation fluid type of the first wellbore for prediction of formation fluid type of the first wellbore before or without formation fluid sampling of the first wellbore.

2. The computer-implemented method according to claim 1, wherein determining at least one of the cumulative rate of change, the instantaneous rate of change, or the combination of the cumulative rate of change and the instantaneous rate of change, of the received formation temperature measurement data over the determined time period of steady-state formation temperature build-up comprises determining
$$\frac{{t}_{n} - {t}_{n - 1}}{\vert {T}_{n} - {T}_{i} \vert}$$
for all points or me received formation temperature measurement data over the determined time period of steady-state formation temperature build-up, wherein tₙ₋₁ and tₙ represent respectively time instants of (n−1)th data point and nth data point of the received formation temperature measurement data over the determined time period of steady-state formation temperature build-up, Tₙ represents nth data point of the received formation temperature measurement data over the determined time period of steady-state formation temperature build-up, and Tᵢ represents the initial data point of the received formation temperature measurement data over the determined time period of steady-state formation temperature build-up.

3. The computer-implemented method according to claim 1, wherein determining at least one of the cumulative rate of change, the instantaneous rate of change, or the combination of the cumulative rate of change and the instantaneous rate of change, of the received formation temperature measurement data over the determined time period of steady-state formation temperature build-up comprises determining
$$\frac{\vert {T}_{n} - {T}_{i} \vert}{{t}_{n} - {t}_{i}}$$
for all data points of the received formation temperature measurement data over the determined time period of steady-state formation temperature build-up, wherein tₙ and tᵢ represent respectively time instants associated with nth data point and initial data point of the received formation temperature measurement data over the determined time period of steady-state formation temperature build-up, Tₙ represents the nth data point of the received formation temperature measurement data over the determined time period of steady-state formation temperature build-up, and Tᵢ represents the initial data point of the received formation temperature measurement data over the determined time period of steady-state formation temperature build-up.

4. The computer-implemented method according to claim 1, further comprising:
before determining the time period of steady-state formation temperature build-up, establishing a baseline cumulative rate of change of water temperature, a baseline instantaneous rate of change of water temperature, a baseline combination of cumulative and instantaneous rates of change of water temperature, a baseline cumulative rate of change of gas temperature, a baseline instantaneous rate of change of gas temperature, and a baseline combination of cumulative and instantaneous rates of change of gas temperature, by using temperature measurement data from a wellbore across a plurality of reservoir intervals, wherein at least one of the plurality of reservoir intervals has a known formation fluid type of water, and at least one of the plurality of reservoir intervals has a known formation fluid type of gas.

5. The computer-implemented method according to claim 4, wherein determining the formation fluid type of the first wellbore based on at least one of the cumulative rate of change, the instantaneous rate of change, or the combination of the cumulative rate of change and the instantaneous rate of change, of the received formation temperature measurement data over the determined time period of steady-state formation temperature build-up comprises at least one of:
comparing the cumulative rate of change of the received formation temperature measurement data with the baseline cumulative rate of change of water temperature and the baseline cumulative rate of change of gas temperature;
comparing the determined instantaneous rate of change of the received formation temperature measurement data with the baseline instantaneous rate of change of water temperature and the baseline instantaneous rate of change of gas temperature; or
comparing the determined combination of the cumulative rate of change and the instantaneous rate of change of the received formation temperature measurement data with the baseline combination of cumulative and instantaneous rates of change of water temperature and the baseline combination of cumulative and instantaneous rates of change of gas temperature.

6. The computer-implemented method according to claim 5, wherein the formation temperature measurement data comprises data from a first temperature measurement point in formation testing at a first depth and data from a second temperature measurement point in formation testing at a second depth, wherein the second depth is deeper or larger than the first depth.

7. The computer-implemented method according to claim 1, wherein determining at least one of the cumulative rate of change, the instantaneous rate of change, or the combination of the cumulative rate of change and the instantaneous rate of change, of the received formation temperature measurement data over the determined time period of steady-state formation temperature build-up comprises determining
$$\frac{\vert {T}_{n} - {T}_{n - 1} \vert}{{t}_{n} - {t}_{n - 1}} or \frac{{t}_{n} - {t}_{n - 1}}{\vert {T}_{n} - {T}_{n - 1} \vert}$$
for all data points of the received formation temperature measurement data over the determined time period of steady-state formation temperature build-up, wherein tₙ and tₙ₋₁ represent respectively time instants associated with nth data point and (n−1)th data point of the received formation temperature measurement data over the determined time period of steady-state formation temperature build-up, and wherein Tₙ and Tₙ₋₁ represent respectively the nth data point and the (n−1)th data point of the received formation temperature measurement data over the determined time period of steady-state formation temperature build-up.

8. A non-transitory, computer-readable medium storing one or more instructions executable by a computer system to perform operations comprising:
receiving formation pressure measurement data and formation temperature measurement data from formation testing of a first wellbore across a reservoir;
determining a time period of steady-state formation temperature build-up during a single formation pressure pre-test of the formation testing;
determining at least one of a cumulative rate of change, an instantaneous rate of change, or a combination of the cumulative rate of change and the instantaneous rate of change, of the received formation temperature measurement data over the determined time period of steady-state formation temperature build-up;
determining formation fluid type of the first wellbore based on at least one of the cumulative rate of change, the instantaneous rate of change, or the combination of the cumulative rate of change and the instantaneous rate of change, of the received formation temperature measurement data over the determined time period of steady-state formation temperature build-up, wherein the formation fluid type comprises one of water and gas; and
providing the determined formation fluid type of the first wellbore for prediction of formation fluid type of the first wellbore before or without formation fluid sampling of the first wellbore.

9. The non-transitory, computer-readable medium according to claim 8, wherein determining at least one of the cumulative rate of change, the instantaneous rate of change, or the combination of the cumulative rate of change and the instantaneous rate of change, of the received formation temperature measurement data over the determined time period of steady-state formation temperature build-up comprises determining
$$\frac{{t}_{n} - {t}_{n - 1}}{\vert {T}_{n} - {T}_{i} \vert}$$
for all data points of the received formation temperature measurement data over the determined time period of steady-state formation temperature build-up, wherein tₙ₋₁ and tₙ represent respectively time instants of (n−1)th data point and nth data point of the received formation temperature measurement data over the determined time period of steady-state formation temperature build-up, Tₙ represents nth data point of the received formation temperature measurement data over the determined time period of steady-state formation temperature build-up, and Tᵢ represents the initial data point of the received formation temperature measurement data over the determined time period of steady-state formation temperature build-up.

10. The non-transitory, computer-readable medium according to claim 8, wherein determining at least one of the cumulative rate of change, the instantaneous rate of change, or the combination of the cumulative rate of change and the instantaneous rate of change, of the received formation temperature measurement data over the determined time period of steady-state formation temperature build-up comprises determining
$$\frac{\vert {T}_{n} - {T}_{i} \vert}{{t}_{n} - {t}_{i}}$$
for all data points of the received formation temperature measurement data over the determined time period of steady-state formation temperature build-up, wherein tₙ and tᵢ represent respectively time instants associated with nth data point and initial data point of the received formation temperature measurement data over the determined time period of steady-state formation temperature build-up, Tₙ represents the nth data point of the received formation temperature measurement data over the determined time period of steady-state formation temperature build-up, and Tᵢ represents the initial data point of the received formation temperature measurement data over the determined time period of steady-state formation temperature build-up.

11. The non-transitory, computer-readable medium according to claim 8, wherein the operations further comprise:
before determining the time period of steady-state formation temperature build-up, establishing a baseline cumulative rate of change of water temperature, a baseline instantaneous rate of change of water temperature, a baseline combination of cumulative and instantaneous rates of change of water temperature, a baseline cumulative rate of change of gas temperature, a baseline instantaneous rate of change of gas temperature, and a baseline combination of cumulative and instantaneous rates of change of gas temperature, by using temperature measurement data from a wellbore across a plurality of reservoir intervals, wherein at least one of the plurality of reservoir intervals has a known formation fluid type of water, and at least one of the plurality of reservoir intervals has a known formation fluid type of gas.

12. The non-transitory, computer-readable medium according to claim 11, wherein determining the formation fluid type of the first wellbore based on at least one of the cumulative rate of change, the instantaneous rate of change, or the combination of the cumulative rate of change and the instantaneous rate of change, of the received formation temperature measurement data over the determined time period of steady-state formation temperature build-up comprises at least one of:
comparing the cumulative rate of change of the received formation temperature measurement data with the baseline cumulative rate of change of water temperature and the baseline cumulative rate of change of gas temperature;
comparing the determined instantaneous rate of change of the received formation temperature measurement data with the baseline instantaneous rate of change of water temperature and the baseline instantaneous rate of change of gas temperature; or
comparing the determined combination of the cumulative rate of change and the instantaneous rate of change of the received formation temperature measurement data with the baseline combination of cumulative and instantaneous rates of change of water temperature and the baseline combination of cumulative and instantaneous rates of change of gas temperature.

13. The non-transitory, computer-readable medium according to claim 12, wherein the formation temperature measurement data comprises data from a first temperature measurement point in formation testing at a first depth and data from a second temperature measurement point in formation testing at a second depth, wherein the second depth is deeper or larger than the first depth.

14. The non-transitory, computer-readable medium according to claim 8, wherein determining at least one of the cumulative rate of change, the instantaneous rate of change, or the combination of the cumulative rate of change and the instantaneous rate of change, of the received formation temperature measurement data over the determined time period of steady-state formation temperature build-up comprises determining
$$\frac{\vert {T}_{n} - {T}_{n - 1} \vert}{{t}_{n} - {t}_{n - 1}} or \frac{{t}_{n} - {t}_{n - 1}}{\vert {T}_{n} - {T}_{n - 1} \vert}$$
for all data points of the received formation temperature measurement data over the determined time period of steady-state formation temperature build-up, wherein tₙ and tₙ₋₁ represent respectively time instants associated with nth data point and (n−1)th data point of the received formation temperature measurement data over the determined time period of steady-state formation temperature build-up, and wherein Tₙ and Tₙ₋₁ represent respectively the nth data point and the (n−1)th data point of the received formation temperature measurement data over the determined time period of steady-state formation temperature build-up.

15. A computer-implemented system, comprising:
one or more computers; and
one or more computer memory devices inter-operably coupled with the one or more computers and having tangible, non-transitory, machine-readable media storing one or more instructions that, when executed by the one or more computers, perform one or more operations comprising:
receiving formation pressure measurement data and formation temperature measurement data from formation testing of a first wellbore across a reservoir;
determining a time period of steady-state formation temperature build-up during a single formation pressure pre-test of the formation testing;
determining at least one of a cumulative rate of change, an instantaneous rate of change, or a combination of the cumulative rate of change and the instantaneous rate of change, of the received formation temperature measurement data over the determined time period of steady-state formation temperature build-up;
determining formation fluid type of the first wellbore based on at least one of the cumulative rate of change, the instantaneous rate of change, or the combination of the cumulative rate of change and the instantaneous rate of change, of the received formation temperature measurement data over the determined time period of steady-state formation temperature build-up, wherein the formation fluid type comprises one of water and gas; and
providing the determined formation fluid type of the first wellbore for prediction of formation fluid type of the first wellbore before or without formation fluid sampling of the first wellbore.

16. The computer-implemented system according to claim 15, wherein determining at least one of the cumulative rate of change, the instantaneous rate of change, or the combination of the cumulative rate of change and the instantaneous rate of change, of the received formation temperature measurement data over the determined time period of steady-state formation temperature build-up comprises determining
$$\frac{{t}_{n} - {t}_{n - 1}}{\vert {T}_{n} - {T}_{i} \vert}$$
for all data points of the received formation temperature measurement data over the determined time period of steady-state formation temperature build-up, wherein tₙ₋₁ and tₙ represent respectively time instants of (n−1)th data point and nth data point of the received formation temperature measurement data over the determined time period of steady-state formation temperature build-up, Tₙ represents nth data point of the received formation temperature measurement data over the determined time period of steady-state formation temperature build-up, and Tᵢ represents the initial data point of the received formation temperature measurement data over the determined time period of steady-state formation temperature build-up.

17. The computer-implemented system according to claim 15, wherein determining at least one of the cumulative rate of change, the instantaneous rate of change, or the combination of the cumulative rate of change and the instantaneous rate of change, of the received formation temperature measurement data over the determined time period of steady-state formation temperature build-up comprises determining
$$\frac{\vert {T}_{n} - {T}_{i} \vert}{{t}_{n} - {t}_{i}}$$
for all data points of the received formation temperature measurement data over the determined time period of steady-state formation temperature build-up, wherein tₙ and tᵢ represent respectively time instants associated with nth data point and initial data point of the received formation temperature measurement data over the determined time period of steady-state formation temperature build-up, Tₙ represents the nth data point of the received formation temperature measurement data over the determined time period of steady-state formation temperature build-up, and Tᵢ represents the initial data point of the received formation temperature measurement data over the determined time period of steady-state formation temperature build-up.

18. The computer-implemented system according to claim 15, wherein the one or more operations further comprise:
before determining the time period of steady-state formation temperature build-up, establishing a baseline cumulative rate of change of water temperature, a baseline instantaneous rate of change of water temperature, a baseline combination of cumulative and instantaneous rates of change of water temperature, a baseline cumulative rate of change of gas temperature, a baseline instantaneous rate of change of gas temperature, and a baseline combination of cumulative and instantaneous rates of change of gas temperature, by using temperature measurement data from a wellbore across a plurality of reservoir intervals, wherein at least one of the plurality of reservoir intervals has a known formation fluid type of water, and at least one of the plurality of reservoir intervals has a known formation fluid type of gas.

19. The computer-implemented system according to claim 18, wherein determining the formation fluid type of the first wellbore based on at least one of the cumulative rate of change or the instantaneous rate of change, or the combination of the cumulative rate of change and the instantaneous rate of change, of the received formation temperature measurement data over the determined time period of steady-state formation temperature build-up comprises at least one of:
comparing the cumulative rate of change of the received formation temperature measurement data with the baseline cumulative rate of change of water temperature and the baseline cumulative rate of change of gas temperature;
comparing the determined instantaneous rate of change of the received formation temperature measurement data with the baseline instantaneous rate of change of water temperature and the baseline instantaneous rate of change of gas temperature; or
comparing the determined combination of the cumulative rate of change and the instantaneous rate of change of the received formation temperature measurement data with the baseline combination of cumulative and instantaneous rates of change of water temperature and the baseline combination of cumulative and instantaneous rates of change of gas temperature.

20. The computer-implemented system according to claim 15, wherein determining at least one of the cumulative rate of change, the instantaneous rate of change, or the combination of the cumulative rate of change and the instantaneous rate of change, of the received formation temperature measurement data over the determined time period of steady-state formation temperature build-up comprises determining
$$\frac{\vert {T}_{n} - {T}_{n - 1} \vert}{{t}_{n} - {t}_{n - 1}} or \frac{{t}_{n} - {t}_{n - 1}}{\vert {T}_{n} - {T}_{n - 1} \vert}$$
for all data points of the received formation temperature measurement data over the determined time period of steady-state formation temperature build-up, wherein tₙ and tₙ₋₁ represent respectively time instants associated with nth data point and (n−1)th data point of the received formation temperature measurement data over the determined time period of steady-state formation temperature build-up, and wherein Tₙ and Tₙ₋₁ represent respectively the nth data point and the (n−1)th data point of the received formation temperature measurement data over the determined time period of steady-state formation temperature build-up.
